(19) United States
(12) Patent Application Publication
(10) Pub. No.: US 2023/0053648 A1
(43) Pub. Date: Feb. 23, 2023

(54) CLOT AND FOREIGN BODY RETRIEVAL SYSTEM AND METHOD FOR USE

(71) Applicant: Yince Loh, Seattle, WA (US)

(72) Inventor: Yince Loh, Seattle, WA (US)

(21) Appl. No.: 18/045,056

(22) Filed: Oct. 7, 2022

Related U.S. Application Data
(63) Continuation of application No. 14/837,787, filed on Aug. 27, 2015, now abandoned.

(51) Int. Cl. A61B 17/221

(52) U.S. Cl. CPC A61B 17/221; A61F 2210/0014; A61F 2230/0091; A61B 2017/22079

(57) ABSTRACT

A retrieval system for thrombus and foreign body removal.

### PRIORITY DATA

This application claims priority from the following US patent applications: this application is a continuation of U.S. application Ser. No. 14/837,787, filed Aug. 27, 2015; which claims the benefit of U.S. Provisional Application Ser. No. 62/056,359, filed on Sep. 26, 2014, and U.S. Provisional Application Ser. No. 62/068,858, filed on Oct. 27, 2014. Each of the above applications is incorporated herein by reference in its entirety, the disclosure of which is incorporated herein in its entirety.

### FIELD OF INVENTION

This invention pertains generally to acute stroke intervention, and more particularly to systems, devices and methods for thrombus or foreign body removal.

### BACKGROUND OF INVENTION

The US is taking an exponentially increasing interest in acute stroke interventions evidenced by its Medicare reimbursement rates, and push for primary and comprehensive stroke center designations. Acute stroke interventions may have significant cost effectiveness and benefit quality of life. US healthcare dollars that are spent on acute interventions are a paltry sum when compared to the lifelong long-term care needs of the victim of a massive, disabling stroke. Medical device companies are aware of this new trend in Medicare reimbursement and are making stroke intervention their new priority. There is a need for a next generation acute stroke intervention device than can be used to treat patients quickly and effectively.

There have been many attempts to develop and treat acute strokes such as improving the blood circulation distal to a cerebral embolus (the clot that causes the stroke). This first started with intravenous infusion of fibrinolysis agents. Physicians have also described ultrasonography or endovascular techniques (from within the blood vessel through catheters and wires inserted in the groin) to include infusion of rat-PA or clot maceration with micro snares or balloons.

A few systems have been designed exclusively for retrieval of the above-described clot. The Merci Retrieval System® was designed and conceived at UCLA by Dr. Y. Pierre Gobin, and manufactured by Concentric Medical, Inc, (now Stryker Medical); this product has been discontinued. The Merci Retrieval System® is comprised of three products: the Merci Retriever®, Merci® Balloon Guide Catheter, and Merci® Microcatheter. The three products are used together in a procedure with the goal of removing a foreign body from an affected vessel. Generally, physicians have used the Merci Retrieval System® to treat clots in brain arteries. Once the location of a clot has been identified using angiography, the Merci® Balloon Guide Catheter is inserted, by way of a small incision in the groin, into the femoral artery. Under x-ray guidance, the Merci® Balloon Guide Catheter is maneuvered up to the carotid artery in the neck, a micro-guidewire and the Merci® Microcatheter are deployed through the Merci® Balloon Guide Catheter and placed just beyond the clot. The Merci® Retriever device is deployed to engage and ensnare the clot. Once the clot is captured, the Merci® Balloon Guide Catheter is inflated to temporarily arrest forward flow while the clot is being withdrawn. The clot is pulled into the Merci® Balloon Guide Catheter and removed from the patient's body. The balloon is deflated and blood flow is restored. The Merci Retrieval System® can also be used to retrieve foreign bodies in the peripheral, coronary, and neuro vasculature.

The Merci Retrieval System® has been studied at UCLA and shown safety and efficacy both with and without additional use of intravenous rat-PA. Unfortunately, however, only about half the patients symptomatic from a vessel occlusion can be effectively recanalized with the Merci Retrieval System®. Clinical improvements are marked in the recanalized group.

The second generation of intracranial thrombectomy device is the aspiration catheter (the “Max” line), manufactured by Penumbra, Inc. Once the location of the clot has been identified using angiography, a larger guiding catheter is inserted, by way of a small incision in the groin, into the femoral artery. Under x-ray guidance, this catheter is maneuvered up to the carotid artery in the neck. An introducing micro-guidewire and a microcatheter are deployed through the aspiration catheter, which are together advanced into the guiding catheter then placed just up against the clot. The physician then removes the introducing micro-guidewire and microcatheter and attaches the aspiration catheter to a suction assembly to aspirate the clot. A separator wire can be used to assist in the debulking of the aspiration catheter tip if it is completely occluded by clot. Alternatively, the aspiration catheter can be removed en bloc with the clot if its tip is occluded. The Penumbra system has been shown to be safe and effective. Similar to the Merci data, not all patients could be recanalized with the system, but the clinical improvements were marked in the recanalized group.

The latest generations of intracranial thrombectomy devices are known as “stent-trievers”. These are non-detachable, fully recoverable stents that are attached distally to a wire. Solitaire is a stent-triever manufactured by eV3, now part of Covidien. Trevo is another stent-triever manufactured by Concentric, now Stryker Medical. The two are similar in concept and overall application and are described as one device herein. Once the location of the clot has been identified using angiography, a balloon guiding catheter is inserted, by way of a small incision in the groin, into the femoral artery. Under x-ray guidance, this catheter is maneuvered up to the carotid artery in the neck. A micro-guidewire and a microcatheter are together advanced into a guiding catheter and then advanced past the clot. The micro-guidewire is removed, and the stent-triever is advanced up to the microcatheter tip. The stent-triever is optimally positioned to engage the clot once unsheathed (pulling the catheter off of the device to let it take its native shape). In its expanded position, the stent-triever temporarily restores flow alongside the clot. Once the clot is engaged by the stent-triever struts, the balloon on the guiding catheter is inflated to temporarily arrest forward flow while the clot is being withdrawn. The clot is pulled into the balloon guiding catheter and completely out of the body. The balloon is then deflated, and blood flow is restored. Both stent-triever systems are safe and effective. However, not all patients can be recanalized with the system; clinical improvements were marked in the recanalized group. There is a need for a device that can ensnare, macerate, and/or separate thromboembolism and other objects from within a patient's cerebral blood vessels.

### BRIEF DESCRIPTION OF INVENTION

An object of the invention is to provide a system, device and method that allows for improved mechanical extraction or maceration of clot (thrombus) or foreign body from within a vessel, either arterial or venous. Another object of the invention is to safely retrieve clots from brain arteries during an acute stroke. Another object of the invention is to provide a system that can be used in conjunction with an aspiration catheter. Another object of the invention is to provide a system that can provide flow around a clot if retrieval is not possible.

### BRIEF DESCRIPTION OF THE SEVERAL VIEWS OF THE DRAWINGS

Other features and advantages of the present invention will become apparent in the following detailed descriptions of the preferred embodiment with reference to the accompanying drawings, of which:

FIG. 1 is a side, prospective view of the retrieval system;

FIG. 2 is a side, perspective view of the retrieval system within a microcatheter;

FIG. 3 is a side view of a of the retrieval system;

FIG. 4 is a side, perspective view of the retrieval system that is partially deployed from a microcatheter;

FIG. 5 is a side, perspective view of the retrieval system being deployed from microcatheter;

FIG. 6 is an end view of the substantially oval rings;

FIG. 7 is a side, perspective view of the retrieval system being deployed from an aspiration catheter;

FIG. 8 is a schematic showing a partially expanded retrieval system engaging a clot/foreign body distally;

FIG. 9 is a schematic showing an expanded retrieval system engaging a clot/foreign body;

FIG. 10A is a schematic of the retrieval system;

FIG. 10B is an end view of a nitinol core;

FIG. 10C is an end view of an out microcatheter assembly.

### DETAILED DESCRIPTION OF THE INVENTION

The present invention, a clot or foreign body retrieval system (“retrieval system”) (300) and methods for use, is described more fully hereinafter with reference to the accompanying drawings, in which preferred embodiments of the invention are shown. This invention may, however, be embodied in many different forms and should not be construed as limited to the embodiments set for herein; rather, these embodiments are provided so that this disclosure will be thorough and complete and will fully convey the scope of the invention to those skilled in the art. Although this device and method are applicable to both clot and foreign body, they are referred to as clot hereinafter.

The inventive system operates on the concept of mechanical thrombectomy (physical retrieval of clot) and addresses hurdles of the Merci Retrieval System® and other prior art devices. These hurdles include: (1) the manner in which a distal device engages the clot; (2) the manner in which the system interacts with the proximal brain vessel anatomy; (3) minimizing the distance of retrieval; (4) reducing vessel trauma when macerating clot under aspiration and (5) improving distal engagement of clot.

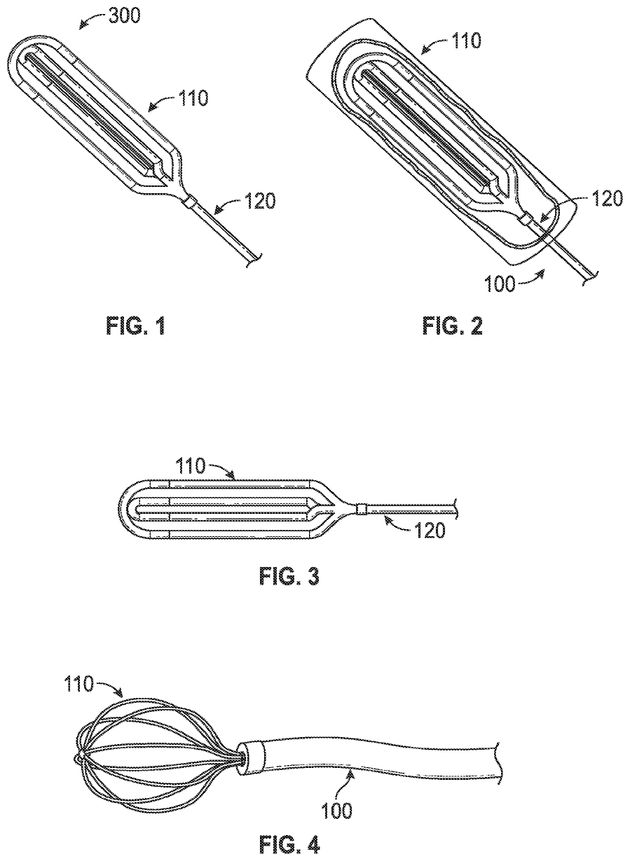

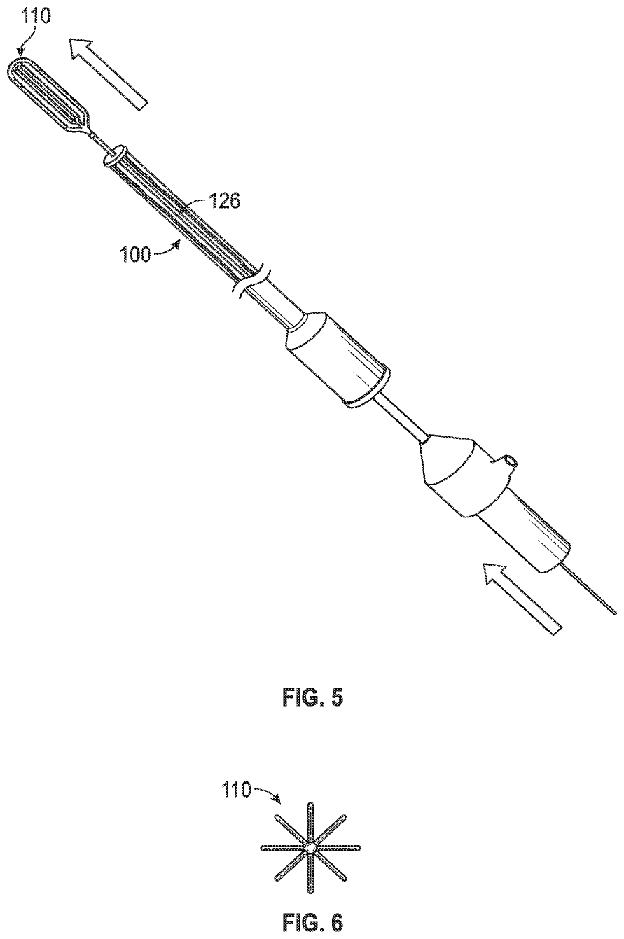

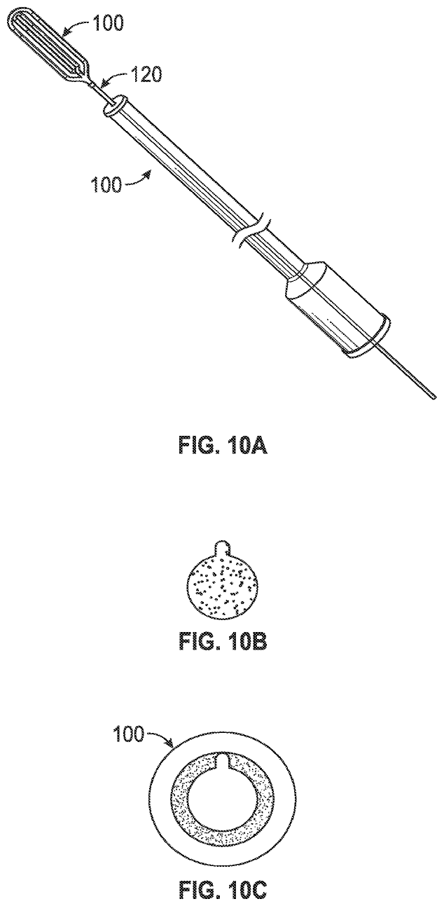

Referring to FIGS. 1 through 10C, the inventive system is a plurality of substantially oval rings 110 attached to a proximal wire 120. The substantially oval rings 110 are moveable between a fully expanded position as illustrated in FIG. 1, and a constrained position as illustrated in FIG. 2. Preferably, the substantially oval rings 110 are made of medical grade nitinol, a nickel and titanium alloy. The substantially oval rings 110 are configured for insertion into an intracorporeal lumen 140 using a microcatheter 200.

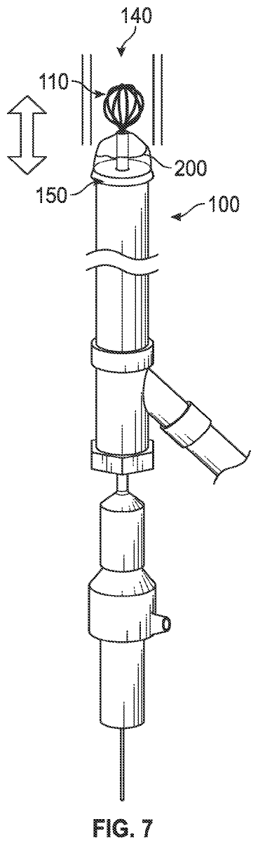

FIG. 7 shows an embodiment of a clot retrieval process according to an embodiment of the inventive system, using a macerative technique. An aspiration catheter 100 is inserted into the intracorporeal lumen proximal to the thrombus. A coaxial microcatheter may be inserted through the clot or between the side of a clot and inner wall of the vessel. The substantially oval rings 110 are then advanced into the microcatheter 200 which is then partially unsheathed back coaxially to expose and deploy the retrieval system 300 tip into the partially expanded position on the distal side of (or within) the clot. Next, the aspiration catheter 100 is advanced into the lumen 140 over the microcatheter 200 and retrieval system 300. Finally, as one unit, the microcatheter 200 and the retrieval system 300 are alternatively pulled (retracted proximally) and pushed (advanced distally) into and out of the aspiration catheter 100 tip.

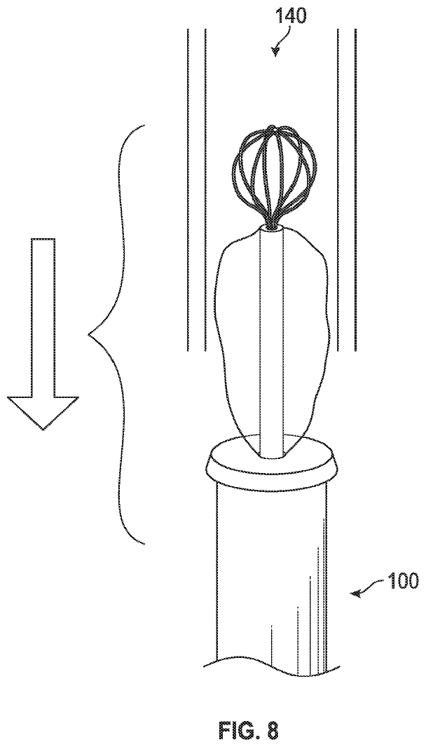

FIG. 8 shows an embodiment of a clot retrieval process according to the present invention using an “en bloc aspiration-retrieval” technique. A guiding catheter is inserted into the intracorporeal lumen 140 and it tip positioned in the lumen of the artery in the neck. Through the guiding catheter, an aspiration catheter 100 is coaxially advanced and positioned in proximity to the clot. A coaxial microcatheter may be inserted through the clot or between the side of a clot and inner wall of the vessel. The retrieval system 300 is then advanced into the microcatheter 200 which is then partially unsheathed back coaxially to expose and deploy the retrieval system 300 tip into the partially expanded position on the distal side of the clot. Next, the aspiration catheter 100 is advanced over the microcatheter 200 and retrieval system 300. The microcatheter 200 and retrieval system 300 are pulled as one unit (retracted proximally) to allow the retrieval system 300 to abut against and/or hook into the distal side of the thrombus and to pull the thrombus proximally so that its proximal side abuts the aspiration catheter 100 tip. The entire system is pulled proximally into the guiding catheter.

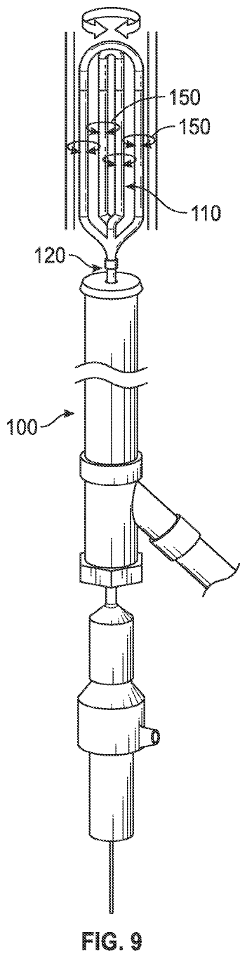

FIG. 9 shows an embodiment of a clot retrieval process according to the present invention, using the fully expanded position. The aspiration catheter 100 is inserted into the intracorporeal lumen 140 proximal to the clot or a balloon guiding catheter is positioned in the lumen of the artery in the neck. A coaxial microcatheter may be inserted through the clot or between the side of a clot and inner wall of the vessel. The retrieval system 300 is advanced into the microcatheter which is then completely unsheathed back coaxially to expose and deploy the retrieval system 300 into the fully expanded position within the extent of the clot. This will provide temporary flow restoration around the clot. Note also that the microcatheter 200 may be removed from the lumen at this time, leaving behind the retrieval system 300. If an aspiration catheter 100 is used, it is advanced over the proximal wire 120. Alternatively, a balloon guiding catheter may be in position in the lumen 140 of the artery in the neck. The retrieval system 300 is pulled proximally into the aspiration catheter 100. Alternatively, the optional balloon on the guiding catheter is inflated, and the retrieval system 300 is pulled proximally into the balloon guiding catheter.

In one embodiment, a rounded, compliant tip may be an extension of the proximal wire 120 to prevent trauma to the distal vessel into which the retrieval system 300 is being inserted. At least one annular ring, band, or similar structure 150 may be circumscribingly, operably attached to the proximal wire 120. Extending outward from the ring or band 150 may be a plurality of resilient, substantially oval rings 110 that are arranged radially and intersect down the longitudinal axis. Preferably, the substantially oval rings 110 are fashioned from nitinol. The substantially oval rings 110 are designed with an “egg-whisker” configuration that produces a multi-faceted interface with the distal side of the clot, and allow for retraction force to be imparted to the clot when the proximal wire 120 is pulled proximally for clot retrieval.

When used with a macerative technique, the aspiration catheter 100 tip abuts the proximal side of the clot during retrieval. This mitigates the “accordion” effect that might otherwise occur. Note also from FIG. 7 that the distance of the initial pull required to dislodge any impacted clot is decreased. Each pull of the retriever transmits force across a short distance, which also mitigates the “accordion” effect.

In this embodiment, preferably, the retrieval system 300 has a 0.014″ to 0.016″ diameter proximal wire 120 and a ring or band 100 from which the plurality of substantially oval rings 110 is welded or otherwise securely attached to the wire; but can be detached using a mechanical or electrolytic mechanism to allow implantation of the retrieval system 300 tip if the clot cannot be removed.

Preferably, the substantially oval rings 110 are moveable from (i) a constrained position where they are forced flush against the proximal wire 120 when inserted into a microcatheter 200, (ii) a partially expanded position where the substantially oval rings 110 are partially deployed and configured for clot maceration during aspiration or clot engagement during “en bloc aspiration-retrieval,” and (iii) a fully expanded position where the substantially oval rings 110 are fully deployed and configured for “clot-wall separation.” Preferably, the extended position is the normal or “memory” position for the substantially oval rings 100. The span of the minor axis of the substantially oval rings 100 (e.g., overall diameter of the distal retriever tip) 15 varies based on the vessel size.

What is claimed is:

1. A device for the retrieval of a clot or foreign body from within a vessel through a catheter comprising:
an elongate wire having a distal end and a proximate end;
a retrieval structure attached to the distal end of the elongate wire;
the retrieval structure comprises a plurality of substantially elliptical wire elements and is deployable from a constrained configuration within the catheter to either a partially expanded configuration or a fully expanded configuration when deployed from the catheter; wherein the plurality of substantially elliptical wire elements, when deployed, form a semi-rigid structure that can engage the clot or foreign body without significant compression.

2. The device of claim 1, wherein, in at least one of the partially and fully expanded configurations, the plurality of wire elements forms a framework having a substantially spheroid shape.

3. The device of claim 1, wherein each of the plurality of wire elements comprises a shape memory alloy material.

4. The device of claim 3, wherein each of the plurality of wire elements comprises a medical grade nitinol alloy.

5. The device of claim 1, wherein the retrieval structure is detachable from the elongate wire.

6. A system for the retrieval of a clot or foreign body from within a vessel comprising:
an elongate wire having a distal end and a proximate end and having a retrieval structure attached to the distal end, wherein the retrieval structure comprises a plurality of substantially elliptical wire elements;
an aspiration catheter having a distal end; and
a microcatheter;
wherein the retrieval structure is disposed in a constrained configuration within the microcatheter and the microcatheter is disposed coaxially within the aspiration catheter, and the retrieval structure is deployable from a constrained configuration within the microcatheter to either a partially expanded configuration or a fully expanded configuration when deployed from the microcatheter at the distal end of the aspiration catheter and wherein the plurality of substantially elliptical wire elements, when deployed, form a semi-rigid structure that can engage the clot or foreign body without significant compression and in conjunction with the aspiration catheter, macerate the clot or foreign body.

7. The system of claim 6, wherein, in at least one of the partially and fully expanded configurations, the plurality of wire elements forms a framework having a substantially spheroid shape.

8. The device of claim 6, wherein each of the plurality of wire elements comprises a shape memory alloy material.

9. The device of claim 8, wherein each of the plurality of wire elements comprises a medical grade nitinol alloy.

10. The device of claim 1, wherein the retrieval structure is detachable from the elongate wire.
